(19) United States
(12) Patent Application Publication
(10) Pub. No.: US 2025/0191698 A1
(43) Pub. Date: Jun. 12, 2025

(54) COMPUTER-READABLE RECORDING MEDIUM STORING COMPRESSION PROGRAM, COMPRESSION METHOD, AND COMPRESSION APPARATUS

(71) Applicant: Fujitsu Limited, Kawasaki-shi (JP)

(72) Inventor: Yosuke OYAMA, Kawasaki (JP)

(73) Assignee: Fujitsu Limited, Kawasaki-shi (JP)

(21) Appl. No.: 18/910,018

(22) Filed: Oct. 9, 2024

(30) Foreign Application Priority Data
Dec. 7, 2023 (JP) 2023-207264

(51) Int. Cl. G16B 50/50

(52) U.S. Cl. CPC G16B 50/50

(57) ABSTRACT

A non-transitory computer-readable recording medium stores a compression program for causing a computer to execute a process including: acquiring protein information represented by an encoded amino acid sequence; compressing the protein information based on a compression policy that defines a relationship between a condition of a compression ratio of the protein information or a condition of time taken when the protein information that has been compressed is decompressed, and a lossless compression function; registering the protein information that has been compressed in a storage device; and when access to the storage device is received, decompressing compressed protein information registered in the storage device.

### CROSS-REFERENCE TO RELATED APPLICATION

This application is based upon and claims the benefit of priority of the prior Japanese Patent Application No. 2023-207264, filed on Dec. 7, 2023, the entire contents of which are incorporated herein by reference.

### FIELD

The embodiment discussed herein is related to a computer-readable recording medium storing a compression program and the like.

### BACKGROUND

A protein, which is a kind of a high molecular weight compound, is composed of 20 kinds of amino acids in a chain form. For example, FASTA format is known as a format in which an amino acid sequence of a protein is described in a code string. In the following description, data of a protein in FASTA format is referred to as “FASTA data” as appropriate.

U.S. Patent Application Publication No. 2020/0294629, Japanese Laid-open Patent Publication No. 2007-193708, Japanese Laid-open Patent Publication No. 2004-240975, and U.S. Patent Application Publication No. 2016/0306919 are disclosed as related art.

### SUMMARY

According to an aspect of the embodiments, a non-transitory computer-readable recording medium stores a compression program for causing a computer to execute a process including: acquiring protein information represented by an encoded amino acid sequence; compressing the protein information based on a compression policy that defines a relationship between a condition of a compression ratio of the protein information or a condition of time taken when the protein information that has been compressed is decompressed, and a lossless compression function; registering the protein information that has been compressed in a storage device; and when access to the storage device is received, decompressing compressed protein information registered in the storage device.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram for describing a breakdown of time taken for a similarity search;

FIG. 2 is a diagram for describing information after compression for some sequences;

FIG. 3 is a functional block diagram illustrating a configuration of a compression apparatus according to the present embodiment;

FIG. 4 is a flowchart illustrating a processing procedure of a compression processing unit according to the present embodiment;

FIG. 5 is a flowchart illustrating a processing procedure of compression processing;

FIG. 6 is a flowchart illustrating a processing procedure of a similarity search unit according to the present embodiment;

FIG. 7 is a flowchart illustrating a processing procedure of decompression processing;

FIG. 8 is a diagram illustrating an example of a .FFdata file and a .FFindex file;

FIG. 9 is a diagram illustrating an example of a case where the compression apparatus is applied to a database in FFindex format;

FIG. 10 is a diagram for describing the size of the .FFdata file according to n;

FIG. 11 is a diagram illustrating a result of applying a compression method described in the present embodiment to a3m and hmm of a Uniclust30 database;

FIG. 12 is a diagram illustrating an example of execution time when hhblits is executed;

FIG. 13 is a diagram illustrating an example of a hardware configuration of a computer that realizes functions similar to those of the compression apparatus of the embodiment;

FIG. 14 is a diagram illustrating an example of FASTA data; and

FIG. 15 is a diagram for describing an example of a similarity search of related art.

### DESCRIPTION OF EMBODIMENTS

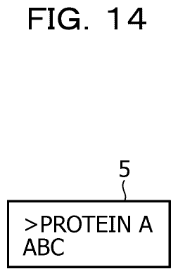

FIG. 14 is a diagram illustrating an example of FASTA data. For example, a protein “PROTEIN A” composed of three amino acids of alanine (“A”), aspartate (“B”), and cystine (“C”) is represented by FASTA data 5.

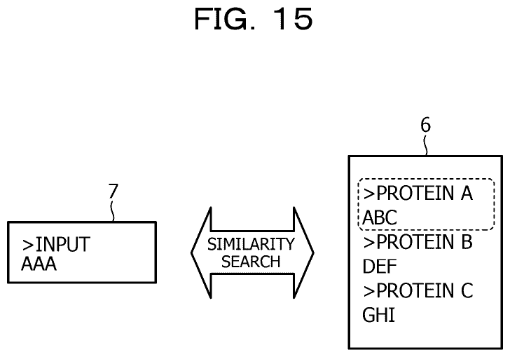

For example, HMMER, HH-suite3, and the like are techniques for performing a similarity search for an input sequence from a protein database that stores FASTA data. FIG. 15 is a diagram for describing an example of a similarity search of related art. In the example illustrated in FIG. 15, pieces of FASTA data of proteins “PROTEIN A”, “PROTEIN B”, and “PROTEIN C” are stored in a protein database 6.

The amino acid sequence of the protein “PROTEIN A” is referred to as “ABC”. The amino acid sequence of the protein “PROTEIN B” is referred to as “DEF”. The amino acid sequence of the protein “PROTEIN C” is referred to as “GHI”.

For example, when “AAA” is designated as an input sequence 7, the protein “PROTEIN A” whose first code of the amino acid sequence matches is searched for.

The actual protein database 6 is a big fantastic database (BFD) or the like, and stores 2.5 billion pieces of FASTA data of proteins. The BFD stores the FASTA data all in an uncompressed state, and the data size is 1.7 TiB.

Since a huge protein database such as the above-described BFD is stored in a network file system or the like having a large capacity but a low speed, the reading speed decreases when a similarity search is performed. For this reason, there is room for improvement in increasing the reading speed in a case where a similarity search is performed.

In one aspect, an object of the present disclosure is to provide a compression program, a compression method, and a compression apparatus that may improve the reading speed in a case where a similarity search is performed.

Hereinafter, an embodiment of a compression program, a compression method, and a compression apparatus disclosed in the present application will be described in detail with reference to the drawings. This disclosure is not limited by the embodiment.

### EMBODIMENT

An example of processing of the compression apparatus according to the present embodiment will be described. The compression apparatus reduces the data size of a protein database by performing lossless compression of data of a protein represented by an encoded amino acid sequence (FASTA data or the like) based on a compression policy set in advance. This makes it possible to store the protein database in a storage device that has a small capacity but is capable of accessing data at high speed.

The storage device that has a small capacity but is accessible at high speed is a central processing unit (CPU) memory, a solid-state drive (SSD), or the like. The lossless-compressed protein database is decompressed when a similarity search is received.

In the following description, a storage device that has a large capacity but a low speed of accessing data is referred to as a “first storage device”. The first storage device is a hard disk drive (HDD), a network file system, or the like. On the other hand, the above-described storage device such as a CPU memory or an SSD is referred to as a “second storage device”. Lossless compression is simply referred to as “compression”. A compressed protein database is referred to as a “compressed database”.

When access to a compressed database stored in the second storage device (a request for a similarity search) is received, the compression apparatus reads the compressed database in the second storage device at high speed, decompresses the compressed database, and performs the similarity search. Consequently, the reading speed in a case of performing a similarity search may be improved.

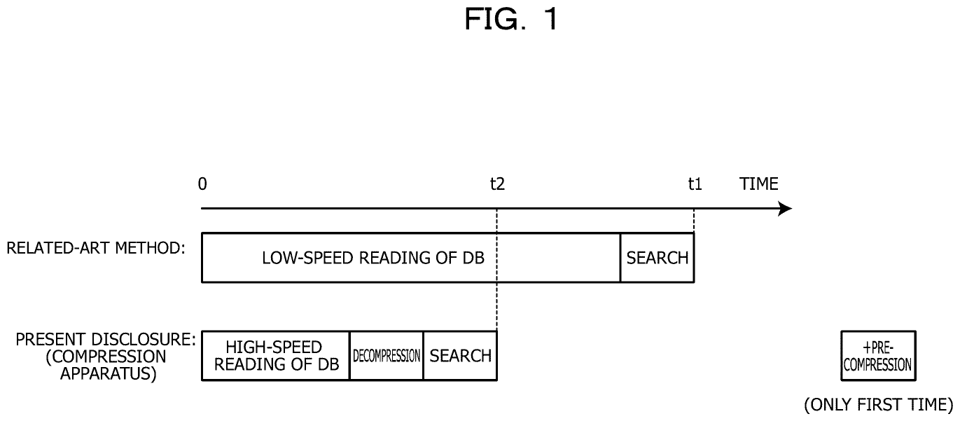

FIG. 1 is a diagram for describing a breakdown of time taken for a similarity search. For example, in the related-art method, when a DB (protein database) is read from the first storage device at low speed and a search is performed, the time taken for the similarity search is t1 time. By contrast, in the compression apparatus of the present disclosure, when a DB (compressed database) is read from the second storage device at high speed, the DB is decompressed, and a search is performed, the time taken for the similarity search is t2 time. Even in consideration of the time of pre-compression executed only at the first time, the time taken for a similarity search of the compression apparatus is shorter than the time taken for a similarity search of the related-art method.

For example, with the compression apparatus according to the present embodiment, although overhead is generated due to decompression of compressed data, the second storage device may be used by reducing the data size of the entire protein database. Consequently, the reading speed in a case of performing a similarity search may be improved.

Subsequently, preconditions of the present embodiment will be described. The preconditions are indicated in the following 1 to 6. 1. A set of byte strings is B, and the number of bytes of byte string b E B is |B| E N (natural number). 2. A set of amino acid sequences of proteins is Bₚ⊂B. In the following description, an amino acid sequence of a protein is referred to as a “sequence”. For example, the sequence “ABC” is “ABC” E Bₚ. 3. Set of lossless compression functions S_{comp} to be applied to each sequence in pre-compression is indicated by formula (1).

$${S}_{comp} = \{ f \} ( f : {B}_{p} \to B , \vert {S}_{comp} \vert > 1 ) \qquad ( 1 )$$

For example, when deflate compression “f_{def}: Bₚ→B” is applied as compression, set of lossless compression functions S_{comp} is indicated by formula (2). id included in formula (2) is identity mapping over Bₚ.

$${S}_{comp} = \{ id , {f}_{def} \} \qquad ( 2 )$$

4. The compression policy is fₚₒₗ: Bₚ→S_{comp}. For example, when compression is performed only in a case where the compression ratio is two times or more, the compression policy is indicated by formula (3). In formula (3), |p| is a length of a sequence. |f_{def}(p)| is a length of a compressed (deflate-compressed) sequence. The compression apparatus performs deflate compression based on formula (3) when the compression ratio is two times or more, and performs identity mapping by id when the compression ratio is less than two times.

$${f}_{pol} ( p ) = \{ \begin{matrix}{f}_{def} & ( \vert p \vert / \vert {f}_{def} ( p ) \vert \geq 2 ) \\ id & ( otherwise )\end{matrix} \qquad ( 3 )$$

5. In the pre-compression, the compression apparatus replaces each sequence p∈Bₚ with the following set of information (c(p), s(p)). For example, (c(p)) is indicated by formula (4). Formula (4) indicates that sequence p is input to a lossless compression function specified by the above-described compression policy f_{def}(p). s(p) is set with a code for identifying the lossless compression function specified by f_{def}(p). For example, when a lossless compression function that performs deflate compression is selected by the compression policy, “1” is set to s(p). When id is selected by the compression policy, “0” is set to s(p). By the value set to s(p), the lossless compression function used when sequence p is compressed is identified.

$$c ( p ) = ( {f}_{pol} ( p ) ) ( p ) \qquad ( 4 )$$

6. In decompression at the time of a similarity search, the compression apparatus restores sequence p by inverse transformation (f_{def}(p))⁻¹ obtained from s(p). For example, the compression apparatus restores sequence p by formula (5).

$$p = {( {f}_{pol} ( p ) )}^{- 1} ( c ( p ) ) \qquad ( 5 )$$

In the above description, a function that performs deflate compression is indicated as a lossless compression function, but the embodiment is not limited to this case. A function such as LZ77/LZ78 compression, Bzip2, LZF, or Snappy may be used. The compression policy is indicated by formula (3), but the embodiment is not limited to this case. The compression apparatus may use any of other compression policies 1 to 4 described below.

Other compression policy 1 will be described. Other compression policy 1 performs compression by lossless compression function f only when the compression ratio is n≥1 time or more, and is indicated by formula (6).

$${f}_{pol} ( p ) = \{ \begin{matrix}f & ( \vert p \vert / \vert f ( p ) \vert \geq n ) \\ id & ( otherwise )\end{matrix} \qquad ( 6 )$$

Other compression policy 2 will be described. Other compression policy 2 uses lossless compression function f₁ or f₂ having a higher compression ratio, and is indicated by formula (7).

$${f}_{pol} ( p ) = \{ \begin{matrix}{f}_{1} & ( \vert {f}_{1} ( p ) \vert \leq \vert {f}_{2} ( p ) \vert ) \\ {f}_{2} & ( otherwise )\end{matrix} \qquad ( 7 )$$

Other compression policy 3 will be described. Other compression policy 3 uses a plurality of lossless compression functions having different compression efficiencies, with a target compression ratio set as n≥1. The plurality of lossless compression functions having different compression efficiencies is indicated by formula (8). Other compression policy 3 is indicated by formula (9) using the plurality of lossless compression functions having different compression efficiencies. However, a general property is assumed in which a lossless compression function having a higher compression ratio takes a longer decompression time. When the size of a database before compression (size A) and the size of a storage desired to be used (size B) are known, the compression apparatus may determine target compression ratio n by the ratio “size A/size B”.

$${\{ {f}_{l} \}}_{l = 1}^{L} \qquad ( 8 )$$

$${f}_{pol} ( p ) = ( {f}_{l} with highest speed satisfying \vert p \vert / \vert {f}_{l} ( p ) \vert \geq n ) \qquad ( 9 )$$

Other compression policy 4 will be described. Other compression policy 4 uses lossless compression function f₁ or f₂ in which the time taken to decompress compressed sequence p is shorter. For example, the time taken to decompress compressed sequence p by lossless compression function f₁ is indicated by formula (10). The time taken to decompress compressed sequence p by lossless compression function f₂ is indicated by formula (11). Other compression policy 4 is indicated as in formula (12) using formula (10) and formula (11).

$${T}_{dec}^{( {f}_{1} )} ( p ) \qquad ( 10 )$$

$${T}_{dec}^{( {f}_{2} )} ( p ) \qquad ( 11 )$$

$${f}_{pol} ( p ) = \{ \begin{matrix}{f}_{1} & ( {T}_{dec}^{( {f}_{1} )} ( p ) \leq {T}_{dec}^{( {f}_{2} )} ( p ) ) \\ {f}_{2} & ( otherwise )\end{matrix} \qquad ( 12 )$$

Other compression policies 1 to 4 have been described above. For example, when a protein database includes a combination of a plurality of different database files, the compression apparatus may apply different compression policies to the respective database files. A compression policy may be applied to some database files. It may be said that a compression policy is information defining a relationship between a condition of a compression ratio of a sequence or a condition of time for decompressing a compressed sequence and a lossless compression function.

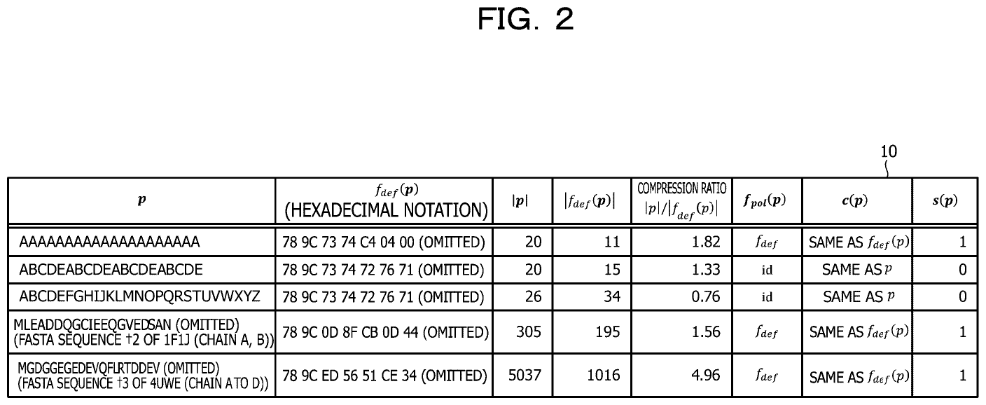

Subsequently, information after compression for some sequences p is illustrated in FIG. 2. FIG. 2 is a diagram for describing information after compression for some sequences p. However, the conditions in the case of compressing sequence p are indicated in the following 1 to 4. 1. f_{def} is a default function that performs deflate compression implemented in zlib v1.2.11. 2. The set of lossless compression functions is the set indicated by formula (2). 3. The compression policy is the compression policy indicated by formula (13). The compression policy indicated by formula (13) corresponds to the compression policy indicated by formula (6) above in which the value of n is set to 1.5. For example, the compression apparatus performs compression when the compression ratio is 1.5 times or more.

$${f}_{pol} ( p ) = \{ \begin{matrix}{f}_{def} & ( \vert p \vert / \vert {f}_{def} ( p ) \vert \geq 1.5 ) \\ id & ( otherwise )\end{matrix} \qquad ( 13 )$$

4. The code of id is “0”, and the code of f_{def} is “1”.

The first line of table 10 in FIG. 2 will be described. Sequence p is “AAAAAAAAAAAAAAAAAAAA”. When such sequence p is compressed by f_{def}, f_{def}(p) is “78 9C 73 74 C4 04 00 (the rest is omitted)”. |p| is “20”, and |f_{def}(p)I is “11”, so the compression ratio is “1.82”. The compression apparatus selects lossless compression function f_{def} based on the compression policy, and replaces sequence p with (c(p), s(p)). C(p) is the same as f_{def}(p). “1” is set to S(p).

The second line of table 10 in FIG. 2 will be described. Sequence p is “ABCDEABCDEABCDEABCDE”. When such sequence p is compressed by f_{def}, f_{def}(p) is “78 9C 73 74 72 76 71 (the rest is omitted)”. |p| is “20”, and |f_{def}(p)| is “15”, so the compression ratio is “1.33”. The compression apparatus selects id based on the compression policy, and replaces sequence p with (c(p), s(p)). C(p) is the same as sequence p. “0” is set to S(p).

The third line of table 10 in FIG. 2 will be described. Sequence p is “ABCDEFGHIJKLMNOPQRSTUVWXYZ”. When such sequence p is compressed by f_{def}, f_{def}(p) is “78 9C 73 74 72 76 71 (the rest is omitted)”. |p| is “26”, and |f_{def}(p)| is “34”, so the compression ratio is “0.76”. The compression apparatus selects id based on the compression policy, and replaces sequence p with (c(p), s(p)). C(p) is the same as sequence p. “0” is set to S(p).

The fourth line of table 10 in FIG. 2 will be described. Sequence p is “MLEADDQGCIEEQGVEDSAN (the rest is omitted)”. When such sequence p is compressed by f_{def}, f_{def}(p) is “78 9C OD 8F CB OD 44 (the rest is omitted)”. |p| is “305”, and |f_{def}(p)| is “195”, so the compression ratio is “1.56”. The compression apparatus selects lossless compression function f_{def} based on the compression policy, and replaces sequence p with (c(p), s(p)). C(p) is the same as f_{def}(p). “1” is set to S(p).

The fifth line of table 10 in FIG. 2 will be described. Sequence p is “MGDGGEGEDEVQFLRTDDEV (the rest is omitted)”. When such sequence p is compressed by f_{def}, f_{def}(p) is “78 9C ED 56 51 CE 34 (the rest is omitted)”. |p| is “5037”, and |f_{def}(p)| is “1016”, so the compression ratio is “4.96”. The compression apparatus selects lossless compression function f_{def} based on the compression policy, and replaces sequence p with (c(p), s(p)). C(p) is the same as f_{def}(p). “1” is set to S(p).

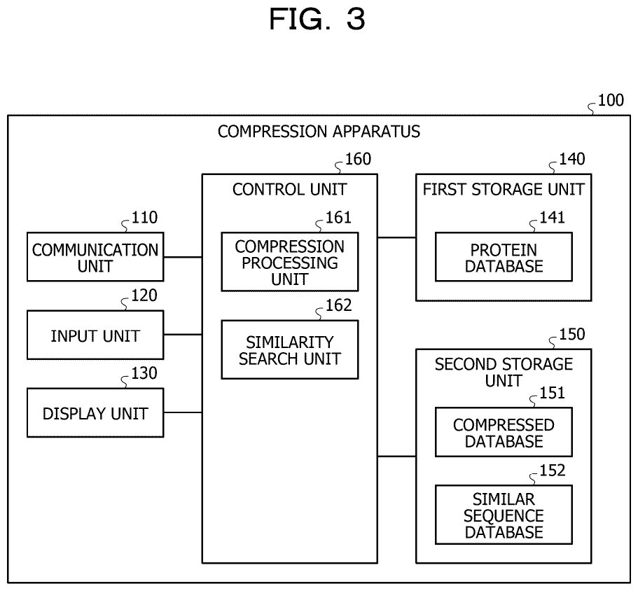

Next, a configuration example of the compression apparatus that executes the above-described processing will be described. FIG. 3 is a functional block diagram illustrating a configuration of the compression apparatus according to the present embodiment. As illustrated in FIG. 3, a compression apparatus 100 includes a communication unit 110, an input unit 120, a display unit 130, a first storage unit 140, a second storage unit 150, and a control unit 160.

The communication unit 110 executes data communication with an external apparatus or the like via a network. The communication unit 110 is a network interface card (NIC) or the like. For example, the compression apparatus 100 may acquire data to be stored in a protein database 141 from an external apparatus.

The input unit 120 is an input device that inputs various types of information to the control unit 160 of the compression apparatus 100. For example, the input unit 120 corresponds to a keyboard, a mouse, a touch panel, or the like. When a similarity search is performed, a user operates the input unit 120 and designates an input sequence.

The display unit 130 is a display device that displays information output from the control unit 160. For example, the display unit 130 displays a search result of a similarity search.

The first storage unit 140 includes the protein database 141. The first storage unit 140 is a storage device that has a large capacity but a low speed of accessing data. The first storage unit 140 is an HDD, a network file system, or the like.

The protein database 141 is a database that stores a plurality of sequences.

The second storage unit 150 includes a compressed database 151 and a similar sequence database 152. The second storage unit 150 is a storage device that has a small capacity but is accessible at high speed. The second storage unit 150 is a CPU memory, an SSD, or the like.

The compressed database 151 is a database that stores compressed sequences.

The similar sequence database 152 is a database that stores sequences similar to an input sequence. The similar sequence database 152 does not have to be stored in the second storage unit 150, and may be stored in the first storage unit 140.

The control unit 160 includes a compression processing unit 161 and a similarity search unit 162. The control unit 160 is a CPU, a graphics processing unit (GPU), or the like.

The compression processing unit 161 acquires a sequence from the protein database 141, and selects a lossless compression function based on a compression policy set in advance. The compression processing unit 161 compresses the sequence by the selected lossless compression function, and stores the compressed sequence in the compressed database 151. For example, the compression processing unit 161 performs compression in which sequence p is replaced with compressed sequence (c(p), s(p)).

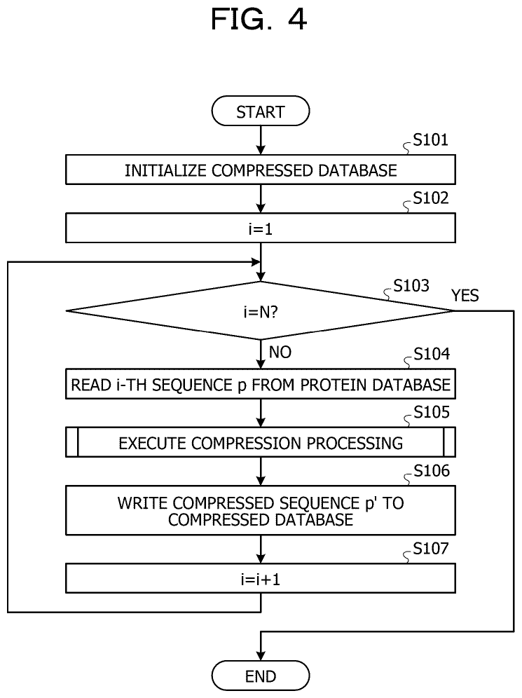

An example of a processing procedure of the compression processing unit 161 will be described. FIG. 4 is a flowchart illustrating a processing procedure of the compression processing unit according to the present embodiment. As illustrated in FIG. 4, the compression processing unit 161 of the compression apparatus 100 initializes the compressed database 151 (step S101).

The compression processing unit 161 sets 1 to i (step S102). When N (a preset numerical value) is set to i (Yes in step S103), the compression processing unit 161 ends the processing. On the other hand, when N is not set to i (No in step S103), the compression processing unit 161 proceeds to step S104.

The compression processing unit 161 reads the i-th sequence p from the protein database 141 (step S104). The compression processing unit 161 executes compression processing for sequence p (step S105). The compression processing unit 161 writes compressed sequence p′ to the compressed database 151 (step S106).

The compression processing unit 161 increases i by 1 (step S107), and proceeds to step S103.

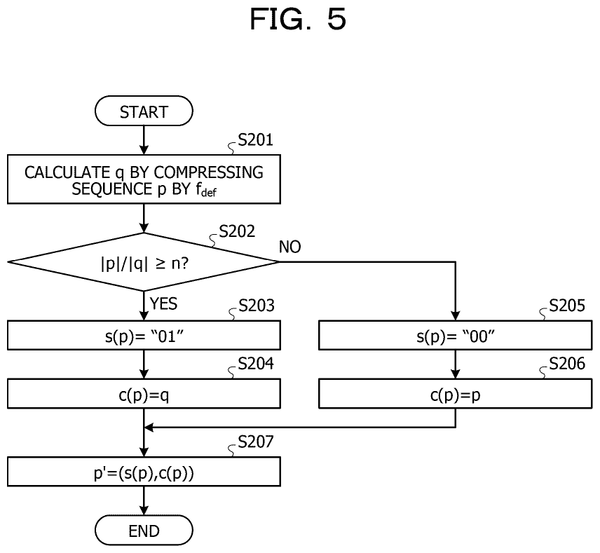

Subsequently, a processing procedure of the compression processing illustrated in step S105 of FIG. 4 will be described. FIG. 5 is a flowchart illustrating a processing procedure of the compression processing. As illustrated in FIG. 5, the compression processing unit 161 calculates q by compressing sequence p by f_{def} (step S201).

When |p|/|q|≥n is satisfied (Yes in step S202), the compression processing unit 161 sets “01” to s(p) (step S203), sets q to c(p) (step S204), and proceeds to step S207.

On the other hand, when |p|/|q| n is not satisfied (No in step S202), the compression processing unit 161 sets “00” to s(p) (step S205), sets p to c(p) (step S206), and proceeds to step S207.

The compression processing unit 161 sets a concatenation of s(p) and c(p) as compressed sequence p′ (step S207).

Returning to the description of FIG. 3. When designation of an input sequence is received from the input unit 120 or the like, the similarity search unit 162 decompresses the compressed sequences in the compressed database 151 and compares the decompressed sequences with the input sequence. The similarity search unit 162 stores decompressed sequences that are similar to the input sequence in the similar sequence database 152. As a similarity search result, the similarity search unit 162 outputs the information of the similar sequence database 152 to the display unit 130 and causes the information to be displayed.

The similarity search unit 162 specifies the lossless compression function used for the compression of sequences based on the numerical value set to s(p) of the compressed sequences, and restores the sequences by inverse transformation of the specified lossless compression function.

The similarity search unit 162 searches for the decompressed sequences that are similar to the input sequence by using a search algorithm implemented in existing software such as HMMER or HH-suite3. For example, the similarity search unit 162 evaluates the degree of matching between a target sequence and the input sequence, and determines that the target sequence is similar to the input sequence when the degree of matching is equal to or greater than a threshold.

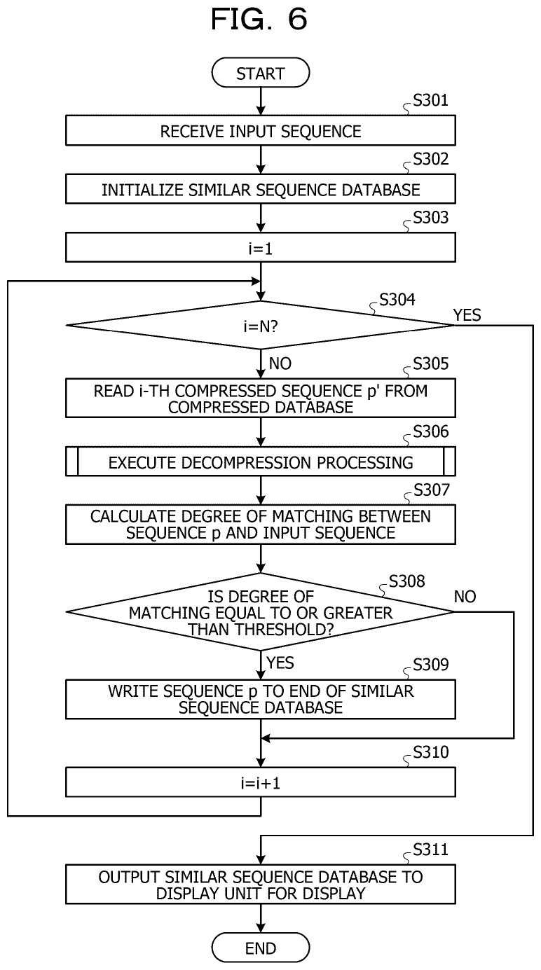

An example of a processing procedure of the similarity search unit 162 will be described. FIG. 6 is a flowchart illustrating a processing procedure of the similarity search unit according to the present embodiment. As illustrated in FIG. 6, the similarity search unit 162 of the compression apparatus 100 receives an input sequence from the input unit 120 (step S301).

The similarity search unit 162 initializes a similar sequence database (step S302). The similarity search unit 162 sets 1 to i (step S303). When N (a preset numerical value) is set to i (Yes in step S304), the similarity search unit 162 outputs the similar sequence database to the display unit 130 and causes the database to be displayed (step S311).

On the other hand, when N is not set to i (No in step S304), the similarity search unit 162 proceeds to step S305. The similarity search unit 162 reads the i-th compressed sequence p′ from the compressed database 151 (step S305).

The similarity search unit 162 executes decompression processing (step S306). The similarity search unit 162 calculates the degree of matching between sequence p and the input sequence (step S307). When the degree of matching is equal to or greater than a threshold (Yes in step S308), the similarity search unit 162 writes sequence p to the end of the similar sequence database (step S309), and proceeds to step S310.

On the other hand, when the degree of matching is not equal to or greater than the threshold (No in step S308), the similarity search unit 162 proceeds to step S310.

The similarity search unit 162 increments i by 1 (step S310), and proceeds to step S304.

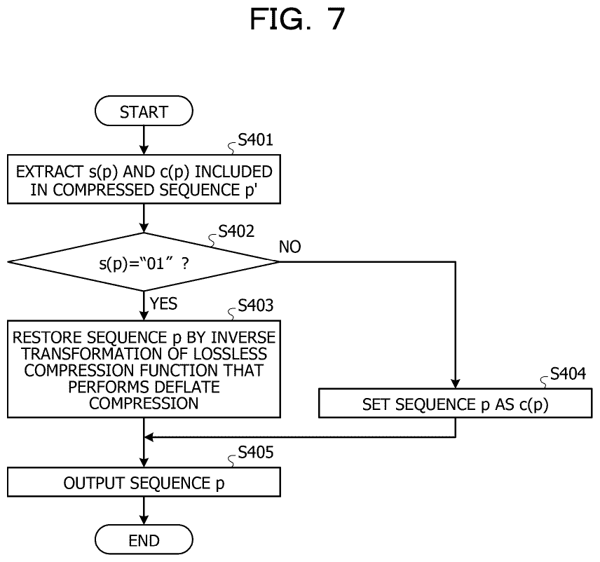

Subsequently, a processing procedure of the decompression processing illustrated in step S306 of FIG. 6 will be described. FIG. 7 is a flowchart illustrating a processing procedure of decompression processing. As illustrated in FIG. 7, the similarity search unit 162 of the compression apparatus 100 extracts s(p) and c(p) included in compressed sequence p′ (step S401).

When the value of s(p) is “01” (Yes in step S402), the similarity search unit 162 restores sequence p by inverse transformation of the lossless compression function that performs deflate compression (step S403), and outputs sequence p (step S405).

On the other hand, when the value of s(p) is not “01” (No in step S402), the similarity search unit 162 sets sequence p as c(p) (step S404), and proceeds to step S405.

A configuration example and an example of a processing procedure of the compression apparatus 100 according to the present embodiment have been described above with reference to FIGS. 3 to 7.

Next, a case will be described in which the above-described compression apparatus 100 is applied to a database in FFindex format. FFindex format is a format used in an actual database.

FFindex is a format for storing one or more pieces of variable-length named binary data, and is composed of a .FFdata file and a .FFindex file. The .FFdata file is a file in which binary data is concatenated and stored with a delimiter ‘\0’. The .FFindex file is a tab-delimited file in which names, offsets (including ‘\0’), and lengths of proteins (including ‘\0’) are listed for each line. Name is the name of a corresponding protein. Offset is position information of the sequence of a corresponding protein (binary data), and indicates an offset from the beginning. Length is the length of the sequence of a corresponding protein.

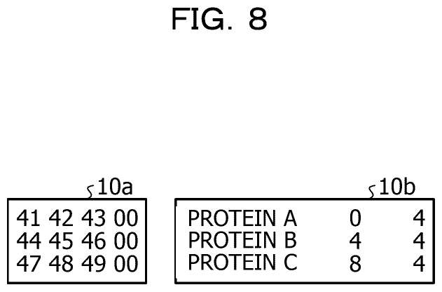

FIG. 8 is a diagram illustrating an example of the .FFdata file and the .FFindex file. The data in the first line of a .FFdata file 10a illustrated in FIG. 8 is data in which the amino acid sequence “ABC” of the protein “PROTEIN A” is concatenated with binary data. “41”, “42”, and “43” are hexadecimal notations of A, B, and C. “00” is a hexadecimal notation of ‘\0’.

In the data in the first line of a .FFindex file 10b, the name “PROTEIN A”, the offset “0”, and the length “4” are set. For example, it is indicated that the sequence with the offset “0” and the length “4” in the .FFdata file 10a is “PROTEIN A”.

The data in the second line of the .FFdata file 10a is data in which the amino acid sequence “DEF” of the protein “PROTEIN B” is concatenated with binary data. “44”, “45”, and “46” are hexadecimal notations of D, E, and F. “00” is a hexadecimal notation of ‘\0’.

In the data in the second line of the .FFindex file 10b, the name “PROTEIN B”, the offset “4”, and the length “4” are set. For example, it is indicated that the sequence with the offset “4” and the length “4” in the .FFdata file 10a is “PROTEIN B”.

The data in the third line of the .FFdata file 10a is data in which the amino acid sequence “GHI” of the protein “PROTEIN C” is concatenated with binary data. “47”, “48”, and “49” are hexadecimal notations of G, H, and I. “00” is a hexadecimal notation of ‘\0’.

In the data in the third line of the .FFindex file 10b, the name “PROTEIN C”, the offset “8”, and the length “4” are set. For example, it is indicated that the sequence with the offset “8” and the length “4” in the .FFdata file 10a is “PROTEIN C”.

As illustrated in FIG. 8, since PROTEIN A, PROTEIN B, and PROTEIN C are all three bytes, they are concatenated as the length of four bytes including ‘\0’.

For example, the conditions in the case where the above-described compression apparatus 100 is applied to a database in FFindex format are indicated in the following 1 to 4. 1. Set of lossless compression functions S_{comp} is the set indicated by formula (2). 2. The compression policy is the policy indicated by formula (6), and n=1.5. 3. As the codes set to s(p), the code of id is “00” and the code of f_{def} is “01”. 4. The data size before compression (excluding ‘\0’) is added to the end of each line in the .FFindex file. By adding the data size before compression, the memory size to be used after decompression may be known before the decompression, and the number of times of memory copying may be reduced.

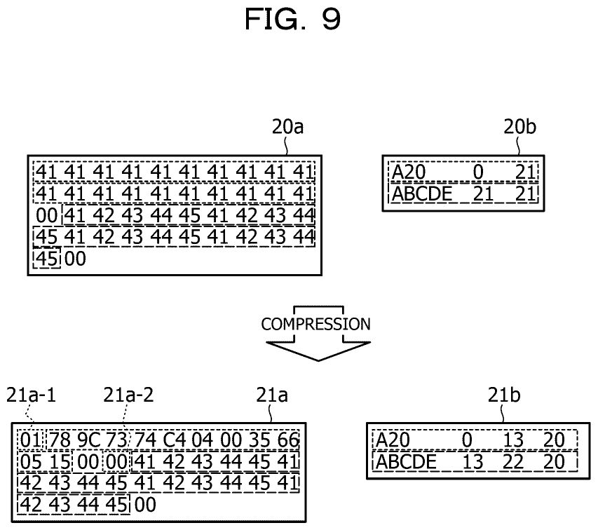

FIG. 9 is a diagram illustrating an example of a case where the compression apparatus is applied to a database in FFindex format. As illustrated in FIG. 9, the .FFdata file before compression is a .FFdata file 20a. The .FFindex file before compression is a .FFindex file 20b. On the other hand, the .FFdata file after compression is a .FFdata file 21a. The .FFindex file after compression is a .FFindex file 21b.

For example, the binary data “41×20” included in the .FFdata file 20a indicates 20 times of repetition of the amino acid code “A” (hereinafter, A20). In the .FFindex file 20b, the name “A20”, the offset “0”, and the length “21” are set as indexes related to A20.

Since the compression ratio for A20 is equal to or greater than 1.5, the compression apparatus 100 selects “f_{def}” as the lossless compression function. The compression apparatus 100 generates c(p)=“78 9C 73 74 C4 04 00 35 66 05 15” by compressing A20 with f_{def}. The compression apparatus 100 sets a set of s(p)=01 (code 21a-1) and c(p)=“78 9C 73 74 C4 04 00 35 66 05 15” in the .FFdata file 21a. The compression apparatus 100 sets the delimiter “00” after c(p)=“78 9C 73 74 C4 04 00 35 66 05 15”.

The compression apparatus 100 registers the data of the first line of the .FFindex file 21b based on the compression result related to A20. For example, the compression apparatus 100 sets the name “A20”, the offset “0”, the length “13”, and the data size before compression “20”.

The binary data “41 42 43 44 45×4” included in the .FFdata file 20a indicates four times of repetition of the code string of amino acids “ABCDE” (hereinafter, ABCDE). In the .FFindex file 20b, the name “ABCDE”, the offset “21”, and the length “21” are set as indexes related to ABCDE.

Since the compression ratio for ABCDE is less than 1.5, the compression apparatus 100 selects “id” as the lossless compression function. The compression apparatus 100 generates c(p)=“41 42 43 44 45×4” by compressing ABCDE with id (identity mapping). The compression apparatus 100 sets a set of s(p)=00 (code 21a-2) and c(p)=“41 42 43 44 45×4” in the .FFdata file 21a. The compression apparatus 100 sets the delimiter “00” after c(p)=“41 42 43 44 45×4”.

The compression apparatus 100 registers the data of the second line of the .FFindex file 21b based on the compression result related to ABCDE. For example, the compression apparatus 100 sets the name “ABCDE”, the offset “13”, the length “22”, and the data size before compression “20”.

As described above, by the compression apparatus 100 performing compression from the .FFdata file 20a to the .FFdata file 21a, 42 bytes is reduced to 35 bytes.

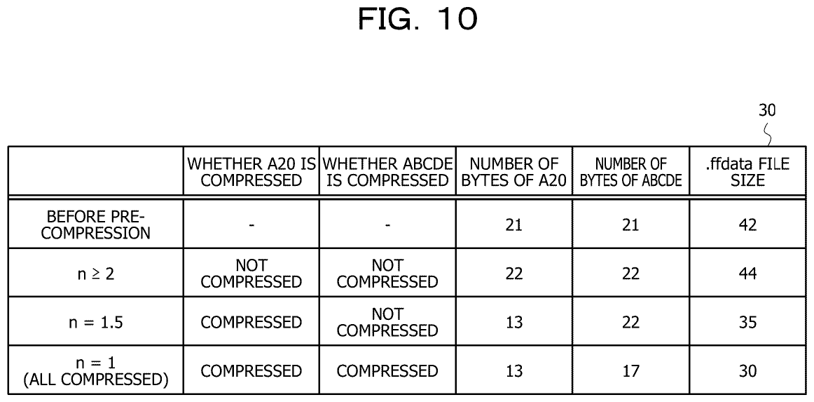

When the value of n is changed to 1, 1.5, or 2 or more, the size of the .FFdata file 20a illustrated in FIG. 9 becomes those illustrated in FIG. 10. FIG. 10 is a diagram for describing the size of the .FFdata file according to n.

The record in the first line of table 30 in FIG. 10 will be described. Before compression, the number of bytes of A20 is “21”, the number of bytes of ABCDE is “21”, and the file size of the .FFdata file 20a is “42”.

The record in the second line of table 30 in FIG. 10 will be described. When n≥2 is set, the compression apparatus 100 selects “id” as the lossless compression function for A20 and ABCDE, and does not compress A20 and ABCDE (performs identity mapping). Consequently, the number of bytes of A20 is “22”, the number of bytes of ABCDE is “22”, and the file size of the .FFdata file 21a after compression is “44”.

The record in the third line of table 30 in FIG. 10 will be described. When n=1.5 is set, the compression apparatus 100 selects “f_{def}” as the lossless compression function for A20, and selects “id” as the lossless compression function for ABCDE. The compression apparatus 100 compresses A20 by f_{def}, and the number of bytes of A20 is “13”. ABCDE is not compressed (identity mapping is performed), and the number of bytes is “22”. Consequently, the file size of the .FFdata file 21a after compression is “35”.

The record in the fourth line of table 30 in FIG. 10 will be described. When n=1 is set, the compression apparatus 100 selects “f_{def}” as the lossless compression function for A20 and ABCDE. The compression apparatus 100 compresses A20 by f_{def}, and the number of bytes of A20 is “13”. The compression apparatus 100 compresses ABCDE by f_{def}, and the number of bytes of ABCDE is “17”. Consequently, the file size of the .FFdata file 21a after compression is “30”.

When the value of n is changed to 1, 1.5, or 2, the compression apparatus 100 may output the relationship between the value of n and the file size of the .FFdata file 21a to the display unit 130 and cause the relationship to be displayed. The compression apparatus 100 may also display the capacity of the second storage unit 150.

For example, the capacity of the second storage unit 150 is 35 bytes. Since the file sizes of the .FFdata file 21a before compression and in the case of n≥2 are “42” and “44”, respectively, as described in FIG. 10, the file may not be stored in the second storage unit 150.

Since the file size of the .FFdata file 21a is “35” when n=1.5, the file may be stored in the second storage unit 150.

Since the file size of the .FFdata file 21a is “30” when n=1, the file may be stored in the second storage unit 150. However, when n=1, since ABCDE is compressed as compared with the case of n=1.5, the time cost for decompression in a similarity search increases.

For this reason, it may be said that n=1.5 is the optimum setting when reading from the second storage unit 150 is sufficiently fast. The compression apparatus 100 may highlight the maximum value of n among the values of n with which the file size of the .FFdata file 21a fits within the capacity of the second storage unit 150. For example, when the capacity of the second storage unit 150 is 35 bytes, the compression apparatus 100 may highlight n=1.5.

Next, the effects of the compression apparatus 100 according to the present embodiment will be described. The compression apparatus 100 acquires protein information, and compresses the protein information based on a compression policy. The compression apparatus 100 registers the compressed protein information in the second storage unit 150. When access related to a request for similarity search is received, the compression apparatus 100 decompresses the compressed protein information registered in the second storage unit 150. Consequently, protein information such as a protein database may be stored in the second storage unit 150 that has a small capacity but is capable of accessing data at high speed, and the reading speed in a case where a similarity search is performed may be improved.

For example, the compression policy defines a plurality of lossless compression functions having different compression ratios. The compression apparatus 100 selects a lossless compression function for which compression ratio used when the protein information is compressed is maximum, and compresses the information on the protein based on the selected lossless compression function. Consequently, compression ratio for a protein may be increased.

For example, the compression policy defines that deflate compression is to be executed when the compression ratio is equal to or greater than a threshold, and identity mapping is to be executed when the compression ratio is less than the threshold. The compression apparatus 100 compresses the protein information by selecting deflate compression or identity mapping. Consequently, deflate compression may be performed for information for which compression ratio is equal to or greater than the threshold, and identity mapping may be performed for information for which compression ratio is less than the threshold. Since identity mapping is performed for those with a low compression ratio, the processing time for decompression may be reduced.

For example, the compression policy defines a plurality of lossless compression functions with different time taken for decompressing the compressed protein information. The compression apparatus 100 selects a lossless compression function with which time taken for decompressing the compressed protein information is minimized, and compresses the protein information based on the selected lossless compression function. Consequently, time taken for a similarity search may be further shortened.

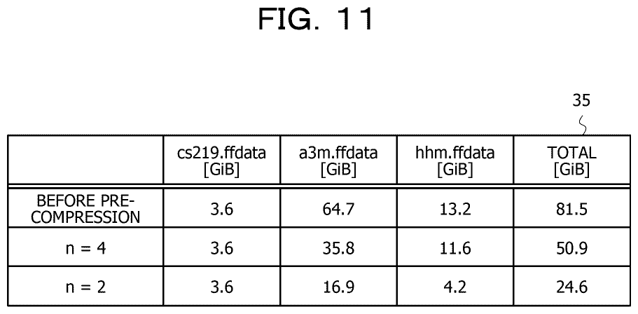

Subsequently, description will be given for a result of evaluating an effect obtained when a similarity search is performed using hhblits (BLAST based on Hidden Markov Model HMM) by applying the compression method of FFindex format described in the present embodiment to a3m and hmm of a Uniclust30 database. FIG. 11 is a diagram illustrating a result of application to a3m and hmm of the Uniclust30 database.

As illustrated in the first line of table 35 in FIG. 11, before compression, the size of cs219.ffdata is “3.6 GiB”, the size of a3 m.ffdata is “64.7 GiB”, the size of hhm.ffdata is “13.2 GiB”, and the total is “81.5 GiB”.

As illustrated in the second line of table 35 in FIG. 11, when n=4, the size of cs219.ffdata is “3.6 GiB”, the size of a3 m.ffdata is “35.8 GiB”, the size of hhm. ffdata is “11.6 GiB”, and the total is “50.9 GiB”.

As illustrated in the third line of table 35 in FIG. 11, when n=2, the size of cs219.ffdata is “3.6 GiB”, the size of a3 m.ffdata is “16.9 GiB”, the size of hhm.ffdata is “4.2 GiB”, and the total is “24.6 GiB”.

As illustrated in FIG. 11, as n is made smaller, the total size is reduced. When n=2, the total size is reduced to about 0.3 times the size before compression.

In this experiment, a similarity search was performed (hhblits was executed) targeting 6R83, which is a protein in the protein data bank (PDB) database included in the OpenProteinSet dataset and had the largest number of hits in the BFD and Uniclust30 database.

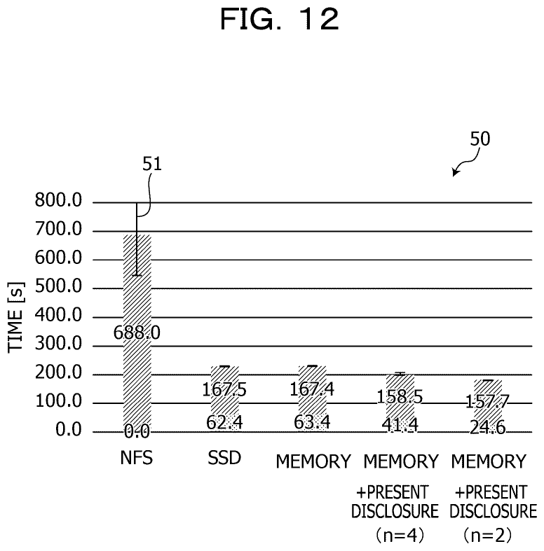

FIG. 12 is a diagram illustrating an example of execution time when hhblits is executed. In graph 50 in FIG. 12, the vertical axis indicates time (execution time), and the horizontal axis indicates condition.

“NFS” indicates a case where all files in Uniclust30 are arranged in the Lustre file system and executed, and the time is “688.0 s”. Error bars 51 indicate the minimum value and maximum value of execution time. Although illustration is omitted, the maximum value corresponding to NFS is “1962.1 s”.

“SSD” indicates a case where all files in Uniclust30 are copied from the Lustre file system to the SSD of the computation node (compression apparatus 100) and then executed, and the time is “167.5 s”. The time taken for copying is “62.4 s”.

“Memory” indicates a case where all files in Uniclust30 are copied to the memory (/dev/shm) of the computation node and then executed, and the time is “167.4 s”. The time taken for copying is “63.4 s”.

“Memory+present disclosure (n=4)” indicates a case where all pre-compressed (n=4) files in Uniclust30 are copied to the memory (/dev/shm) of the computation node and then executed, and the time is “158.5 s”. The time taken for copying is “41.4 s”.

“Memory+present disclosure (n=2)” indicates a case where all pre-compressed (n=2) files in Uniclust30 are copied to the memory (/dev/shm) of the computation node and then executed, and the time is “157.7 s”. The time taken for copying is “24.6 s”.

From the above experimental result, the following may be said. Only with “NFS”, the execution time of hhblits is very long. This is because the database is read via a network, which is a bottleneck in terms of performance.

The hhblits execution time of “memory+present disclosure” has substantially the same performance as those of “SSD” and “memory”. This is because the process other than the reading of a3m and hhm (and decompression in a case of using the present disclosure) becomes a bottleneck by the use of a high-speed storage.

Since the Uniclust30 database has a smaller data size as n is smaller in the present disclosure, the copying time is reduced.

When the upper limit of the size of SSD and memory that may be used for holding the database is assumed to be 64 GiB, “SSD” and “memory” may not be executed, and, without the present disclosure, the low-speed “NFS” has to be used. On the other hand, when the present disclosure is used, copying to the memory is made possible in both cases of n=2 and n=4, and the execution speed of hhblits is significantly increased. For example, in “memory+present disclosure (n=2)”, the speed is increased by 4.36 times (only the execution time of hhblits) or 3.77 times (overall) as compared with “NFS”.

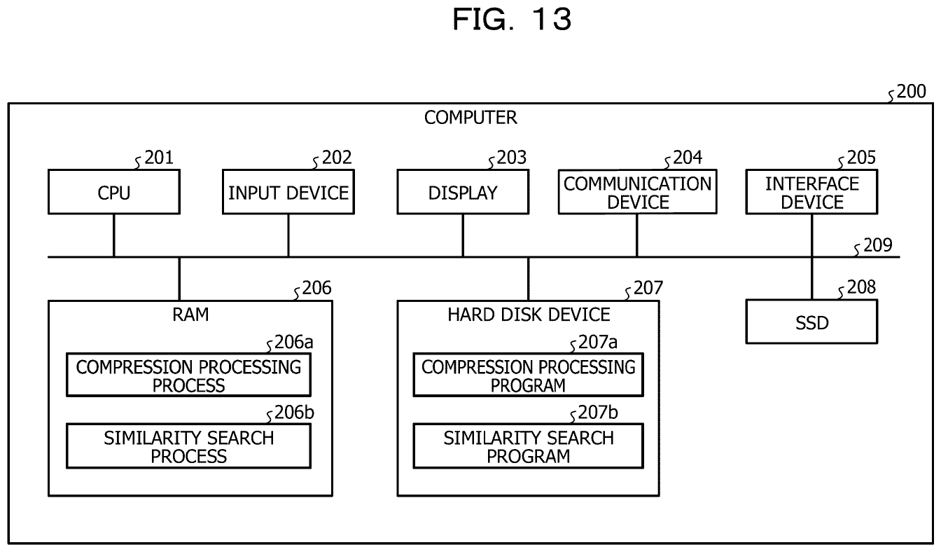

Next, an example of a hardware configuration of a computer that realizes functions similar to those of the compression apparatus 100 described above will be described. FIG. 13 is a diagram illustrating an example of a hardware configuration of a computer that realizes functions similar to those of the compression apparatus of the embodiment.

As illustrated in FIG. 13, a computer 200 includes a CPU 201 that executes various types of arithmetic processing, an input device 202 that receives input of data from a user, and a display 203. The computer 200 includes a communication device 204 that exchanges data with an external apparatus or the like via a wired or wireless network, and an interface device 205. The computer 200 includes a random-access memory (RAM) 206 that temporarily stores various types of information, a hard disk device 207, and an SSD 208 that is accessible at high speed. Each of the devices 201 to 208 is coupled to a bus 209.

The hard disk device 207 includes a compression processing program 207a and a similarity search program 207b. The CPU 201 reads each of the programs 207a and 207b and loads the programs to the RAM 206.

The compression processing program 207a functions as a compression processing process 206a. The similarity search program 207b functions as a similarity search process 206b.

Processing of the compression processing process 206a corresponds to the processing of the compression processing unit 161. Processing of the similarity search process 206b corresponds to the processing of the similarity search unit 162.

Each of the programs 207a and 207b does not have to be stored in the hard disk device 207 from the beginning. For example, each program is stored in a “portable physical medium”, such as a flexible disk (FD), a compact disc read-only memory (CD-ROM), a Digital Versatile Disc (DVD), a magneto-optical disk, or an integrated circuit (IC) card, to be inserted into the computer 200. The computer 200 may read and execute each of the programs 207a and 207b.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a compression program for causing a computer to execute a process comprising:
acquiring protein information represented by an encoded amino acid sequence;
compressing the protein information based on a compression policy that defines a relationship between a condition of a compression ratio of the protein information or a condition of time taken when the protein information that has been compressed is decompressed, and a lossless compression function;
registering the protein information that has been compressed in a storage device; and
when access to the storage device is received, decompressing compressed protein information registered in the storage device.

2. The non-transitory computer-readable recording medium according to claim 1, wherein
the compression policy defines a plurality of lossless compression functions for which the compression ratio is different, and in the compressing of the protein information, a lossless compression function that has a highest compression ratio when the protein information is compressed is selected, and the protein information is compressed based on a selected lossless compression function.

3. The non-transitory computer-readable recording medium according to claim 2, wherein
the compression policy defines that deflate compression is to be executed when the compression ratio is equal to or greater than a threshold and identity mapping is to be executed when the compression ratio is less than the threshold, and in the compressing, the deflate compression or the identity mapping is selected and the protein information is compressed.

4. The non-transitory computer-readable recording medium according to claim 1, wherein
the compression policy defines a plurality of lossless compression functions with different time taken when the protein information that has been compressed is decompressed, and in the compressing of the protein information, a lossless compression function with shortest time taken when the protein information that has been compressed is decompressed is selected and the protein information is compressed based on a selected lossless compression function.

5. A compression method for causing a computer to execute a process comprising:
acquiring protein information represented by an encoded amino acid sequence;
compressing the protein information based on a compression policy that defines a relationship between a condition of a compression ratio of the protein information or a condition of time taken when the protein information that has been compressed is decompressed, and a lossless compression function;
registering the protein information that has been compressed in a storage device; and
when access to the storage device is received, decompressing compressed protein information registered in the storage device.

6. The compression method according to claim 5, wherein
the compression policy defines a plurality of lossless compression functions for which the compression ratio is different, and in the compressing of the protein information, a lossless compression function that has a highest compression ratio when the protein information is compressed is selected, and the protein information is compressed based on a selected lossless compression function.

7. The compression method according to claim 6, wherein
the compression policy defines that deflate compression is to be executed when the compression ratio is equal to or greater than a threshold and identity mapping is to be executed when the compression ratio is less than the threshold, and in the compressing, the deflate compression or the identity mapping is selected and the protein information is compressed.

8. The compression method according to claim 5, wherein
the compression policy defines a plurality of lossless compression functions with different time taken when the protein information that has been compressed is decompressed, and in the compressing of the protein information, a lossless compression function with shortest time taken when the protein information that has been compressed is decompressed is selected and the protein information is compressed based on a selected lossless compression function.

9. A compression apparatus comprising:
a memory; and
a processor coupled to the memory and configured to:
acquire protein information represented by an encoded amino acid sequence;
compress the protein information based on a compression policy that defines a relationship between a condition of a compression ratio of the protein information or a condition of time taken when the protein information that has been compressed is decompressed, and a lossless compression function;
register the protein information that has been compressed in a storage device; and
when access to the storage device is received, decompress compressed protein information registered in the storage device.

10. The compression apparatus according to claim 9, wherein
the compression policy defines a plurality of lossless compression functions for which the compression ratio is different, and in an process to compress the protein information, a lossless compression function that has a highest compression ratio when the protein information is compressed is selected, and the protein information is compressed based on a selected lossless compression function.

11. The compression apparatus according to claim 10, wherein
the compression policy defines that deflate compression is to be executed when the compression ratio is equal to or greater than a threshold and identity mapping is to be executed when the compression ratio is less than the threshold, and in the process to compress, the deflate compression or the identity mapping is selected and the protein information is compressed.

12. The compression apparatus according to claim 9, wherein
the compression policy defines a plurality of lossless compression functions with different time taken when the protein information that has been compressed is decompressed, and in an process to compress the protein information, a lossless compression function with shortest time taken when the protein information that has been compressed is decompressed is selected and the protein information is compressed based on a selected lossless compression function.
